(19) United States
(12) Patent Application Publication
(10) Pub. No.: US 2026/0335605 A1
(43) Pub. Date: Oct. 29, 2026

(54) An Introducer with Lubricating Cap

(71) Applicant: Bard Peripheral Vascular, Inc., Franklin Lakes, NJ (US)

(72) Inventors: Bryon Ray Densley, Rochester, MN (US); Diana Fiumefreddo, Salt Lake City, UT (US); Danielle Mariah Pompa, Bountiful, UT (US); Ian N. Thomas, West Bountiful, UT (US); Brenda L. F. Fedor, Holladay, UT (US)

(21) Appl. No.: 19/106,086

(22) PCT Filed: Aug. 25, 2022
(86) PCT No.: PCT/US2022/041573

§ 371 Date: Feb. 24, 2025

(51) Int. Cl. A61M 25/06; A61M 39/00; A61M 39/20

(52) U.S. Cl. CPC A61M 25/0662; A61M 39/20; A61M 2039/0036

(57) ABSTRACT

Disclosed herein is a medical device assembly that includes a cap applied to a connector of the medical device. The cap can provide a lubricant to a valve of the medical device. The lubricant can be disposed within in a cap cavity together with a wicking material. A tube of the cap can provide a flow path for the lubricant to flow to the valve. The cap may transition the valve between a closed state and an open state. A distal projection of the cap may extend through an opening of the valve. A method of manufacture of the medical device is disclosed that includes placing the lubricant and wicking material within the cap cavity and applying the cap to the medical device. The cap may define a fluid tight seal with the connector and may also define a sterile barrier for the connector.

### BACKGROUND

Medical devices such as catheters, introducers, IV sets may include valves incorporated therein to selectively allow and prevent fluid flow through the medical device. Some valves may include a septum having an opening that may be transitioned between a closed state and an open state. Septum based valves within connectors of medical devices provide for easy opening of the valve via engagement of the separate medical device and self-closure of the valve upon disengagement of the separate medical device. Furthermore, septum based valves are inexpensive and easily manufactured. However, septum based valves can experience degradation (e.g., drying out) over time especially during storage of the medical device before use. The degradation may cause the valve to not open as intended or not reseal upon closure of the valve. The degradation can limit the shelf life for the medical device which can contribute to lack of inventory of the medical device or expiration of the medical device before use.

The devices and methods disclosed herein address the forgoing.

### SUMMARY

Disclosed herein is an introducer, that according to some embodiments, includes (i) an elongate member defining an elongate lumen extending between a distal end configured for placement within a blood vessel and a proximal end; (ii) a hub coupled with the elongate member at the proximal end, where the hub is configured for use outside a patient; (iii) a connector coupled with the hub opposite the elongate member, where the connector defines a connector lumen; and (iv) a valve coupled between the connector and the hub. The valve is configured to selectively transition between (i) a closed state, where fluid flow is prevented between the connector lumen and the elongate lumen, and (ii) an opened state where fluid flow is allowed between the connector lumen and the elongate lumen.

The introducer further includes a cap coupled with the connector, where the cap, according to some embodiments, includes (i) a circumferential wall coupled with a proximal end wall; (ii) a medial transverse wall extending inward from the circumferential wall; and (iii) a tubular member coupled with the medial transverse wall, where the tubular member defines a tubular lumen in fluid communication with an opening extending through the medial transverse wall, and where the circumferential wall, the proximal end wall, the medial transverse wall, and the tubular member define a cap cavity. A wicking material is disposed within the cap cavity, and a cap lubricant dispersed within the wicking material. The tubular member extends into the connector lumen toward the valve so that a distal end of the tubular lumen is disposed adjacent the valve.

In some embodiments, the wicking material is disposed within the tubular lumen.

In some embodiments, the cap defines a fluid tight seal with the connector. In some embodiments, the tubular member defines a fluid tight seal with the connector.

In some embodiments, the cap defines a sterile barrier for the connector.

In some embodiments, the tubular member engages the valve to transition the valve to the opened state.

In some embodiments, the tubular member includes a distal projection extending distally beyond a distal end of the tubular member, and the distal projection extends through an opening of the valve. In some embodiments, the distal projection defines a blade shape.

In some embodiments, the valve includes a valve lubricant disposed within the opening of the valve, where the valve lubricant is configured to define a lubrication between opposing edge surfaces of the valve. In some embodiments, the cap lubricant is configured to maintain the lubrication between the opposing edge surfaces of the valve during storage of the introducer. In some embodiments, the valve lubricant is impregnated within the material of the valve, and the cap lubricant is configured to inhibit exudation of the valve lubricant from the material of the valve during storage of the introducer.

In some embodiments, the cap is configured for removal from the connector by rotating the cap with respect to the connector. In some embodiments, the connector includes external threads, and the circumferential wall includes corresponding internal threads configured to engage the external threads to secure the cap to the connector.

Also disclosed herein is a medical cap, that according to some embodiments, includes (i) a circumferential wall coupled with a proximal end wall; (ii) a medial transverse wall extending inward from the circumferential wall; and (iii) a tubular member coupled with the medial transverse wall, where the tubular member defines a tubular lumen in fluid communication with an opening extending through the medial transverse wall, and where the circumferential wall, the proximal end wall, the medial transverse wall, and the tubular member define a cap cavity. A wicking material is disposed within the cap cavity, and a cap lubricant dispersed within the wicking material. The cap is configured to (i) couple with a connector of a medical device, and (ii) transition a valve of the medical device between a closed state and an opened state.

In some embodiments of the cap, the wicking material is disposed within the tubular lumen.

In some embodiments of the cap, the cap is configured to define a fluid tight seal with the connector.

In some embodiments of the cap, the tubular member is configured to define a fluid tight seal with the connector.

In some embodiments of the cap, the cap is configured to define a sterile barrier for the connector.

In some embodiments of the cap, the tubular member is configured to engage the valve to transition the valve to the opened state when the cap is coupled with connector.

In some embodiments of the cap, the tubular member includes a distal projection extending distally beyond a distal end of the tubular member, and the distal projection is configured to extend through an opening of the valve when the cap is coupled with connector.

In some embodiments of the cap, the distal projection defines a blade shape.

In some embodiments of the cap, the cap lubricant is configured to maintain a lubrication between opposing edge surfaces of the valve during storage of the medical device.

In some embodiments of the cap, the cap lubricant is configured to inhibit exudation of a valve lubricant impregnated within a material of the valve during storage of the medical device.

In some embodiments of the cap, the cap is configured for removal from the connector by rotating the cap with respect to the connector.

In some embodiments of the cap, the circumferential wall includes internal threads corresponding to external threads of the connector includes, and the internal threads are configured to engage the external threads to secure the cap to the connector.

In some embodiments of the cap, the medical device is an introducer.

Also disclosed herein is a method for manufacturing a medical device assembly, that, according to some embodiments, includes providing a medical device. The medical device includes (i) an elongate member defining an elongate lumen extending between a distal end configured for placement within a blood vessel and a proximal end; (ii) hub coupled with the elongate member at the proximal end, where the hub configured for use outside a patient; (iii) a connector coupled with the hub opposite the elongate member, where the connector defines a connector lumen; and (iv) a valve coupled between the connector and the hub. The valve is configured to selectively transition between (i) a closed state where fluid flow is prevented between the connector lumen and the elongate lumen, and (ii) an opened state where fluid flow is allowed between the connector lumen and the elongate lumen.

The method further includes providing a cap configured for coupling with the connector, the cap including (i) a circumferential wall; (ii) a medial transverse wall extending inward from the circumferential wall; and (iii) a tubular member coupled with the medial transverse wall, where the tubular member defines a tubular lumen in fluid communication with an opening extending through the medial transverse wall, and further where the circumferential wall, the medial transverse wall, and the tubular member define a cap cavity.

The method further includes (i) placing a wicking material within the cap cavity; (ii) dispersing a cap lubricant into the wicking material; and (iii) applying the cap to the connector.

In some embodiments of the method, placing the wicking material within the cap cavity includes placing the wicking material within the tubular lumen.

In some embodiments, the method further includes coupling a proximal end wall to a proximal end of the circumferential wall to seal a proximal end of the cap cavity after placing the wicking material within the cap cavity and after dispensing the cap lubricant into the cap cavity.

In some embodiments of the method, applying the cap to the connector includes defining a fluid tight seal between the cap and the connector.

In some embodiments of the method, applying the cap to the connector includes establishing a sterile barrier for the connector.

In some embodiments of the method, applying the cap to the connector includes transitioning the valve to the opened state.

In some embodiments of the method, applying the cap to the connector includes inserting a distal projection of the tubular through an opening of the valve.

In some embodiments of the method, the valve includes a valve lubricant disposed within the opening of the valve, the valve lubricant configured to define a lubrication between opposing edge surfaces of the valve, and applying the cap to the connector includes providing the cap lubricant to the valve to maintain the lubrication between the opposing edge surfaces of the valve.

In some embodiments of the method, applying the cap to the connector includes rotating the cap with respect to the connector.

In some embodiments of the method, the medical device is an introducer.

These and other features of the concepts provided herein will become more apparent to those of skill in the art in view of the accompanying drawings and following description, which describe particular embodiments of such concepts in greater detail.

### DRAWINGS

FIG. 1 illustrates a perspective view of a medical device assembly, in accordance with some embodiments.

FIG. 2 illustrates a perspective view of the cap of the medical device assembly of FIG. 1, in accordance with some embodiments.

FIG. 3A illustrates a cross-sectional front view of the medical device assembly of FIG. 1, in accordance with some embodiments.

FIG. 3B illustrates a cross-sectional detail view of valve portion of the medical device assembly depicted in FIG. 3A, in accordance with some embodiments.

FIG. 4 is a block diagram of a method of manufacturing of the medical device assembly of FIG. 1, in accordance with some embodiments.

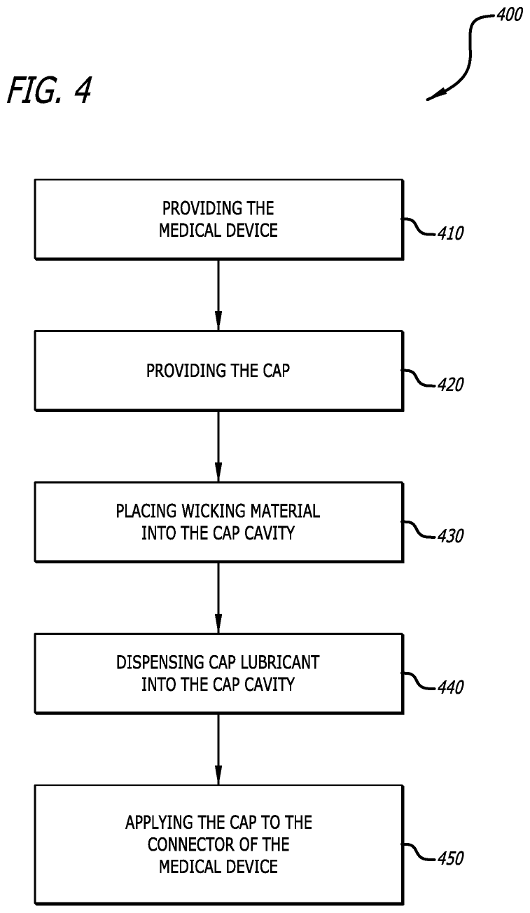

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,”“front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the proximal end of introducer may be defined as the end closest to the practitioner during use of the introducer. The distal end is the end opposite the proximal end, along the longitudinal direction of the introducer, such as the end inserted within the patient.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Unless defined otherwise, all technical and scientific terms used herein have the same meaning as commonly understood by those of ordinary skill in the art.

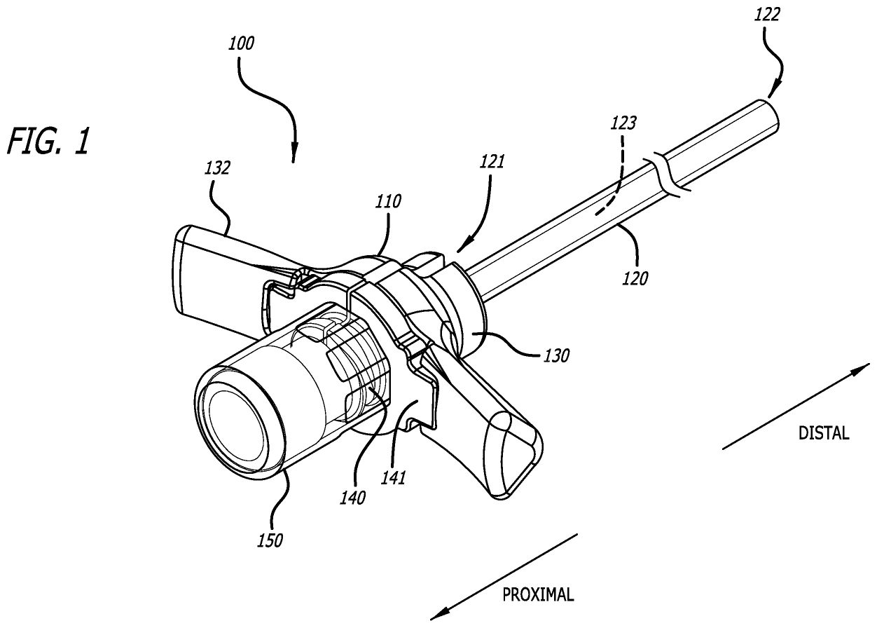

FIG. 1 is a perspective view of a medical device assembly, according to some embodiments. The medical device assembly (assembly) 100 generally include a medical device (device) 110 having a cap 150 coupled therewith. The device 110 may be any type of a tubular medical device such as an intravascular device (e.g., a catheter) or an infusion device (e.g., an IV set). In the illustrated embodiment, the device 110 is an introducer. The device generally includes an elongate member 120 defining a proximal end 121, a distal end 122 and an elongate lumen 123 extending along the elongate member 120 between the proximal end 121 and the distal end 122. A hub 130 of the device 110 is coupled with the elongate member 120 defining at the proximal end 121. A portion of the elongate member 120 including the distal end is configured for insertion into a blood vessel of a patient and the hub 130 is disposed outside the patient during use of the device 110. The hub 130 may include a pair of wings 132 to enable handing of the device 110 (more specifically the hub 130) by a clinician during use of the device 110. The hub 130 includes a connector 140. In some embodiments, the connector 140 includes a flange 141 configured for coupling the connector 140 with a body portion of the hub 130. The cap 150 is coupled with the device 110 so as to cover the connector 140. The cap 150 may be coupled with the device 110 during storage of the device before use, and the cap 150 may be decoupled from the device 110 during use. In some embodiments, the cap 150 may define a sterile barrier for the connector 140. In the illustrated embodiment, the cap 150 may be configured to extend a shelf life of the device 110 as described below.

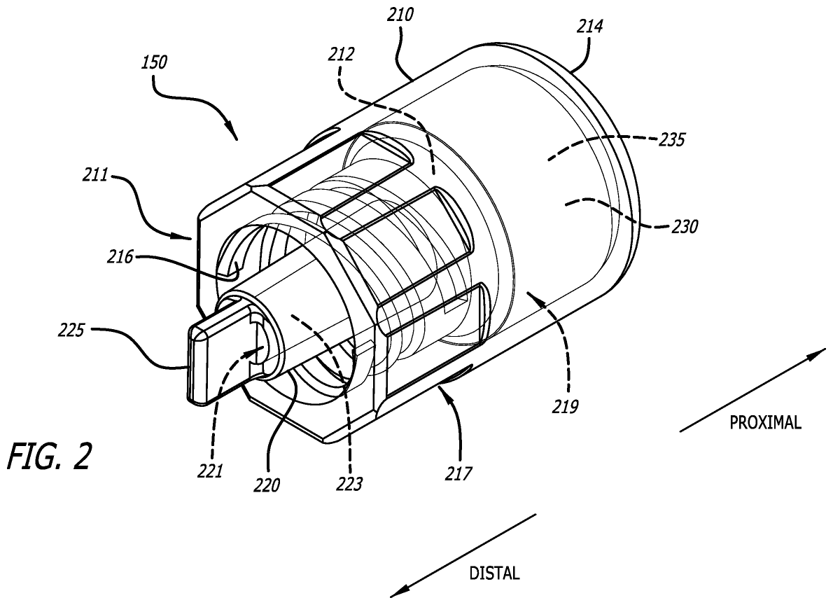

FIG. 2 is a perspective view of the cap 150, according to some embodiments. The cap 150 is shown as transparent in FIG. 2 for illustrative purposes. The cap 150 may generally be formed from a biocompatible thermoplastic material via the injection molding process. The cap 150 is generally configured for coupling with the connector 140 so that the cap 150 remains secured to the connector 140 until the cap 150 is removed by the clinician.

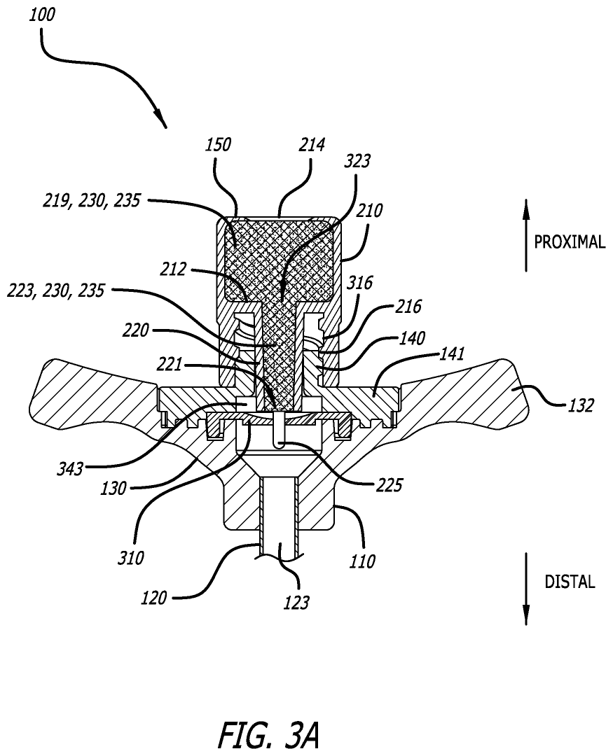

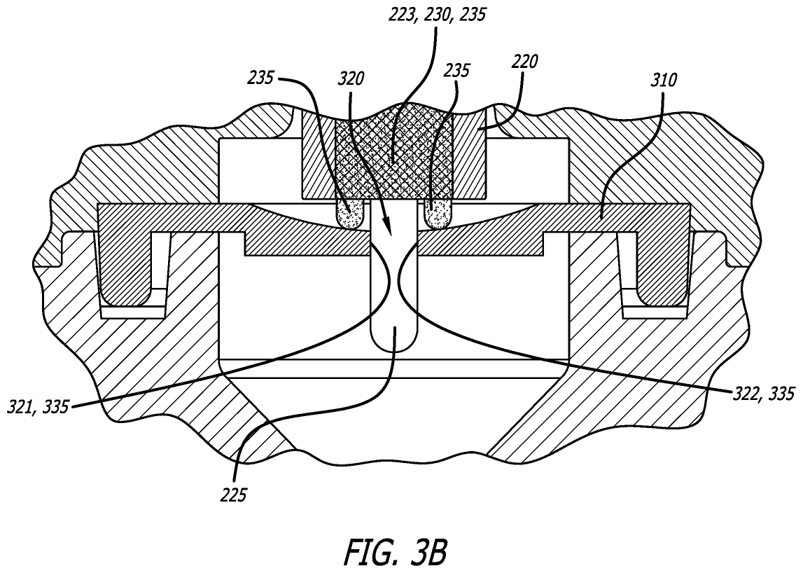

In the illustrated embodiment, the cap 150 includes a circumferential wall 210 coupled with a proximal end wall 214. In some embodiments, the proximal end wall 214 may be formed separately from the circumferential wall 210 and subsequently coupled therewith. A medial transverse wall 212 extends inward from the circumferential wall 210. A tubular member 220 is coupled with the medial transverse wall 121. The tubular member 220 defines a tubular lumen 223 extending between a distal opening 221 and an opening 323 (see FIG. 3A) extending through the medial transverse wall 212. The circumferential wall 210, the proximal end wall 214, the medial transverse wall 212, and the tubular member 220 define a cap cavity 219. The tubular member 220 may extend distally beyond the distal end 211 of the circumferential wall 210 A wicking material 230 is disposed within the cap cavity 219. In some embodiments, the wicking material 230 may fill the cap cavity 219. In other embodiments, the wicking material 230 may fill only of portion of the cap cavity, such as the tubular lumen 223, for example. Exemplary wicking materials may include fibrous materials, such as a wool, polyester, polypropylene, nylon, linen, rayon, or cotton.

A cap lubricant 235 is disposed within the cap cavity 219. In some embodiments, the cap lubricant 235 may be dispersed within the wicking material 230. The cap 150 is generally configured to contain the cap lubricant 235 and allow the cap lubricant 235 to exit the cap cavity 219 via the distal opening 221 of the tubular member 220. The cap lubricant 235 is generally configured to maintain the lubrication of a valve (see FIGS. 3A, 3B) of the device 110 as described below. In some embodiments, the lubricant 235 may be a silicone lubricant.

In the illustrated embodiment, the cap 150 includes a distal projection 225 coupled with the tubular member 220 so as to extend distally beyond the distal opening 221 of the tubular member 220. The distal projection 225 may be configured to engage the valve as further described below. In the illustrated embodiment, the distal projection 225 defines a blade shape. In some embodiments, the opposite edges of the blade shaped distal projection 225 may be sharp. The sharp edges (not shown) may be configured to minimize strain of the valve material when the distal projection 225 is disposed with the opening of the valve. In other embodiments, the distal projection 225 may define a shape other than the blade shape, such as a pin shape, for example. In some embodiments, the distal projection 225 may include the cap lubricant 235 disposed on the surfaces of the distal projection 225 so that the distal projection 225 may dispense cap lubricant 235 to the valve. The distal projection 225 may formed of the same material as the cap 150, i.e., a thermal plastic material, such as Delrin, or polyacetal, for example. In some embodiments, the distal projection 225 may be omitted.

The circumferential wall 210 and the tubular member 220 generally define annular cavity configured for receiving the connector 140 therein. Internal threads 216 extend inward from the circumferential wall 210 within the annular cavity. The internal threads 216 are configured to threadingly engage corresponding external threads of the connector 140 to secure the cap 150 to the connector 140.

In some embodiments, the cap 150 may include gripping features 217 disposed on an outside surface of the circumferential wall 210 to enable the clinician to handle and manipulate the cap 150 during use. The gripping features 217 may include one or more of protrusions, depressions, ribs, troughs, or a polygonal shape.

In the illustrated embodiment, the cap 150 is a component of the assembly 100. In other embodiments, the cap 150 may be a stand-alone medical device. In some instances, there may be a need to couple a new cap 150 to a medical device from which an original cap had been removed. In such other embodiments, the cap 150 may be disposed within a package (not shown), such as a bag, for example, where the package defines a sterile barrier for the cap 150 during storage.

FIG. 3A is a cross-sectional front view of the assembly 100, according to some embodiments. Shown are the wicking material 230 and the cap lubricant 235 disposed within the cap cavity 219 including the tubular lumen 223. Corresponding external threads 316 of the connector 140 are threadingly engaged with the internal threads 216 of the cap 150. The tubular member 220 is disposed within a connector lumen 343 of the connector 140.

In some embodiments, the cap 150 may define a fluid tight seal with the connector 140. By way of one example, the tubular member 220 may include a tapered outer surface configured to define a tapered fit with the connector 140, where the tapered fit defines a fluid tight seal with the connector 140. By way of another example, the circumferential wall 210 may define a fluid tight seal with the connector 140.

The device 110 further includes a valve 310 in the form of a septum. The valve 310 is configured to selectively transition between (i) a closed state, where fluid flow is prevented between the connector lumen 343 and the elongate lumen 123, and (ii) an opened state where fluid flow is allowed between the connector lumen 343 and the elongate lumen 123. In some embodiments, the transition from the closed state to the opened state may be due to interaction of the valve 310 with the tubular member 220 when the cap 150 is coupled with the connector 140. In some embodiments, the tubular member 220 may cause the valve 310 to deflect away from the closed state to the opened state. The valve 310 may be formed of an elastomeric material, such as silicone, EPDM, or polyurethane, for example.

FIG. 3B illustrates a detailed cross-sectional view the valve 310 along with associated components. The valve 310 includes a valve opening 320. When the valve 310 is in the closed state, the valve opening 320 is in a closed state so that fluid flow through the valve opening 320 is prevented. Similarly, when the valve 310 is the opened state, the valve opening 320 is in an opened state so that fluid flow through the valve opening 320 is allowed. The valve opening 320 includes opposing edge surfaces 321, 322. When the valve 310 is the closed state, the opposing edge surfaces 321, 322 are in contact with each other to the define the closed state. When the valve 310 is the closed state, the opposing edge surfaces 321, 322 are separated from each other to the define the opened state.

In some instances, the opposing edge surfaces 321, 322 may bond to each other (e.g., reknit) so that the valve 310 is prevented from transitioning to the opened state. To combat this bonding of the opposing edge surfaces 321, 322, the valve 310 includes a valve lubricant 335 that is at least disposed on the opposing edge surfaces 321, 322 to lubricate the valve 310. The valve lubricant 335 may also facilitate sliding displacement of a separate medical device, such as a guidewire or stylet, for example through the valve opening 320. In some embodiments, the valve lubricant 335 may also be impregnated within the valve material.

In some instances, the valve lubricant 335 may exude from the valve 310 over time (e.g., a storage time or shelf life) so that the benefits of the valve lubricant are dimensioned rendering the device 110 unfit for use. When the cap 150 is applied to the connector 140, cap lubricant 235 may flow from the tubular lumen 223 to the valve 310 (including the opposing edge surfaces 321, 322) so as to supplement the lubrication of the valve 310 and thereby, extend the shelf life of the device 110. In some embodiments, the cap lubricant 235 may inhibit exudation of the valve lubricant 335 from the valve material. In some embodiments, the valve lubricant 335 and the cap lubricant 235 may be the same or similar.

In the illustrated embodiment, the distal projection 225 is disposed through the valve opening 320 of the valve 310. The disposition of the distal projection 225 within the valve opening 320 may facilitate the flow of the cap lubricant 235 to the opposing edge surfaces 321, 322. The valve 310 may be configured to self-transition to the closed state upon removal of the distal projection 225 from the valve opening 320.

A method of manufacturing the medical device assembly may include all or subset of the following steps, processes or actions. According to some embodiments, the method of manufacturing includes providing the medical device (block 410), where the medical device includes the elongate member, the hub coupled with the elongate member, the connector coupled with the hub and the valve coupled between the connector and the hub. Providing the medical device may also include applying the valve lubricant to the valve.

The method of manufacturing may further include providing the cap (block 420), where the cap includes the cap cavity as defined by the circumferential wall, the medial transverse wall, and the tubular member.

The method may further include placing the wicking material into the cap cavity (block 430). According to one embodiment, placing the wicking material includes inserting the wicking material proximally through the tubular lumen. According to one embodiment, placing the wicking material includes placing the wicking material into the cap cavity via an open proximal end of the cap and then coupling the proximal end wall to the circumferential wall.

The method may further include dispensing the cap lubricant into the cap cavity (block 440). According to one embodiment, dispensing the cap lubricant into the cap cavity includes dispersing the cap lubricant into the wicking material. According to one embodiment, dispensing the cap lubricant includes dispensing the cap lubricant proximally through the through the tubular lumen. According to another embodiment, dispensing the cap lubricant includes dispensing the cap lubricant into the cap cavity via an open proximal end of the cap and then coupling the proximal end wall to the circumferential wall.

The method may further include applying the cap to the connector (block 450). Applying the cap to the connector may also include defining a fluid tight seal between the cap and the connector. Applying the cap to the connector may also include establishing a sterile barrier for the connector. Applying the cap to the connector may also include transitioning the valve to the opened state. Applying the cap to the connector may also include inserting the distal projection of the tubular member through an opening of the valve. Applying the cap to the connector may also include providing the cap lubricant to the valve. Applying the cap to the connector may also include rotating the cap with respect to the connector.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An introducer, comprising:
an elongate member defining an elongate lumen extending between a distal end configured for placement within a blood vessel and a proximal end;
a hub coupled with the elongate member at the proximal end, the hub configured for use outside a patient;
a connector coupled with the hub opposite the elongate member, the connector defining a connector lumen;
a valve coupled between the connector and the hub, the valve configured to selectively transition between:
a closed state where fluid flow is prevented between the connector lumen and the elongate lumen, and
an opened state where fluid flow is allowed between the connector lumen and the elongate lumen; and
a cap coupled with the connector, the cap comprising:
a circumferential wall coupled with a proximal end wall;
a medial transverse wall extending inward from the circumferential wall; and
a tubular member coupled with the medial transverse wall, the tubular member defining a tubular lumen in fluid communication with an opening extending through the medial transverse wall, wherein the circumferential wall, the proximal end wall, the medial transverse wall, and the tubular member define a cap cavity;
a wicking material disposed within the cap cavity; and
a cap lubricant disposed within the cap cavity,
wherein the tubular member extends into the connector lumen toward the valve so that a distal end of the tubular lumen is disposed adjacent the valve.

2. The introducer according to claim 1, wherein the wicking material is disposed within the tubular lumen.

3. The introducer according to claim 1, wherein the cap defines a fluid tight seal with the connector.

4. The introducer according to claim 1, wherein the cap defines a sterile barrier for the connector.

5. The introducer according to claim 1, wherein the tubular member engages the valve to transition the valve to the opened state.

6. The introducer according to claim 1, wherein:
the tubular member includes a distal projection extending distally beyond a distal end of the tubular member, and
the distal projection extends through an opening of the valve.

7. The introducer according to claim 6, wherein the distal projection defines a blade shape.

8. The introducer according to claim 6, wherein the valve includes a valve lubricant disposed within the opening of the valve, the valve lubricant configured to define a lubrication between opposing edge surfaces of the valve.

9. The introducer according to claim 8, wherein the cap lubricant is configured to maintain the lubrication between the opposing edge surfaces of the valve during storage of the introducer.

10. The introducer according to claim 1, wherein the cap is configured for removal from the connector by rotating the cap with respect to the connector.

11. The introducer according to claim 1, wherein the connector includes external threads, and wherein the circumferential wall includes corresponding internal threads configured to engage the external threads to secure the cap to the connector.

12. A medical cap, comprising:
a circumferential wall coupled with a proximal end wall,
a medial transverse wall extending inward from the circumferential wall; and
a tubular member coupled with the medial transverse wall, the tubular member defining a tubular lumen in fluid communication with an opening extending through the medial transverse wall, wherein the circumferential wall, the proximal end wall, the medial transverse wall, and the tubular member define a cap cavity;
a wicking material disposed within the cap cavity; and
a cap lubricant disposed within the cap cavity, wherein the cap is configured to couple with a connector of a medical device, and wherein the cap is configured to transition a valve of the connector between a closed state and an opened state.

13. The cap according to claim 12, wherein the wicking material is disposed within the tubular lumen.

14. The cap according to claim 12, wherein the cap is configured to define a fluid tight seal with the connector.

15. The cap according to claim 12, wherein the cap is configured to define a sterile barrier for the connector.

16. The cap according to claim 12, wherein the tubular member is configured to engage the valve to transition the valve to the opened state when the cap is coupled with connector.

17. The cap according to claim 12, wherein:
the tubular member includes a distal projection extending distally beyond a distal end of the tubular member, and
the distal projection is configured to extend through an opening of the valve when the cap is coupled with connector.

18. The cap according to claim 17, wherein the distal projection defines a blade shape.

19. The cap according to claim 17, wherein:
the opening of the valve includes opposing edge surfaces, and
the cap lubricant is configured to maintain a lubrication between the opposing edge surfaces of the opening during storage of the medical device.

20. The cap according to claim 12, wherein the cap is configured for removal from the connector by rotating the cap with respect to the connector.

21. The cap according to claim 12, wherein the circumferential wall includes internal threads corresponding to external threads of the connector, and wherein the internal threads are configured to engage the external threads to secure the cap to the connector.

22. The cap according to claim 12, wherein the medical device is an introducer.

23-32. (canceled)
